(19) United States
(12) Patent Application Publication
(10) Pub. No.: US 2010/0252053 A1
(43) Pub. Date: Oct. 7, 2010

(54) METHODS AND APPARATUS FOR REDUCTION OF LACTATE

(76) Inventors: Dena Petty Garner, Isle of Palms, SC (US); Mark Allen Roettger, Lake Elmo, MN (US); Henry Daniel Cross, III, Murrells Inlet, SC (US); Paul Anthony Broadbent, Mill Valley, CA (US); Paul C. Belvedere, Edina, MN (US)

Correspondence Address: Clise, Billion & Cyr, P.A., 605 U.S. Highway 169, Suite 300, Plymouth, MN, 55441, US

(21) Appl. No.: 12/571,184

(22) Filed: Sep. 30, 2009

Related U.S. Application Data
(63) Continuation-in-part of application No. 10/460,886, filed on Jun. 12, 2003.
Application No. 10/460,886 is a continuation-in-part of application No. 09/657,421, filed on Sep. 8, 2000, now Pat. No. 6,626,180.
Application No. 09/657,421 is a continuation-in-part of application No. 12/484,216, filed on Aug. 26, 2009.
Application No. 12/484,216 is a continuation of application No. PCT/US2009/047295, filed on Jun. 14, 2009.
Application No. PCT/US2009/047295 is a continuation-in-part of application No. 10/460,886, filed on Jun. 12, 2003.
Application No. 10/460,886 is a continuation-in-part of application No. 09/657,421, filed on Sep. 8, 2000, now Pat. No. 6,626,180.
Application No. 09/657,421 is a continuation-in-part of application No. 12/484,223, filed on Jun. 14, 2009.
Application No. 12/484,223 is a continuation-in-part of application No. 10/460,886, filed on Jun. 12, 2003.
Application No. 10/460,886 is a continuation-in-part of application No. 09/657,421, filed on Sep. 8, 2000, now Pat. No. 6,626,180.
(60) Provisional application No. 61/101,411, filed on Sep. 30, 2008.
(60) Provisional application No. 61/132,588, filed on Jun. 19, 2008.

(51) Int. Cl. A61F 5/00

(52) U.S. Cl. 128/845

(57) ABSTRACT

Oral apparatus and methods to inhibit the increase of lactate levels during physical and/or mental exertion are disclosed. The oral apparatus include at least a first bite pad and a second bite pad. The first bite pad and the second bite pad are each configured to be positioned between at least two of the molars of a user. The bite pads are generally configured to direct the posterior of the lower mandible downward and/or forward in response to a clenching force exerted by a user. When worn during physical and/or mental exertion, the oral apparatus can reduce the accumulation of lactate normally generated by the physical and/or mental exertion without the oral apparatus.

### CROSS REFERENCE TO RELATED APPLICATIONS

The present application claims benefit and priority to U.S. Prov. Pat. Appl. No. 61/101,411, filed on Sep. 30, 2008 which is hereby incorporated by reference into the present disclosure.

The present application also claims benefit and priority as a continuation-in-part of co-pending U.S. patent application Ser. No. 10/460,886, filed on Jun. 12, 2003 and now published as U.S. Pat. Pub. No. 2004/0250817, which is a continuation-in-part of U.S. patent application Ser. No. 09/657,421, filed on Sep. 8, 2000 and now issued as U.S. Pat. No. 6,626,180 each of which is hereby incorporated by reference into the present disclosure.

The present application also claims benefit and priority as a continuation-in-part of co-pending U.S. patent application Ser. No. 12/484,216, filed on Aug. 26, 2009, which is both a continuation of a patent application filed under the Patent Cooperation Treaty having Int'l Appl. No. PCT/US2009/047295, filed on Jun. 14, 2009, which claims the benefit and priority of U.S. Prov. Pat. App. Ser. No. 61/132,588, filed on Jun. 19, 2008, and is a continuation-in-part of U.S. patent application Ser. No. 10/460,886, filed on Jun. 12, 2003 and now published as U.S. Pat. Pub. No. 2004/0250817, which is a continuation-in-part of U.S. patent application Ser. No. 09/657,421, filed on Sep. 8, 2000 and now issued as U.S. Pat. No. 6,626,180 each of which is hereby incorporated by reference into the present disclosure.

The present application also claims benefit and priority as a continuation-in-part of co-pending U.S. patent application Ser. No. 12/484,223, filed on Jun. 14, 2009, which is a continuation in part of co-pending U.S. patent application Ser. No. 10/460,886, filed on Jun. 12, 2003 and is now published as U.S. Pat. Pub. No. 2004/0250817, which is a continuation-in-part of U.S. patent application Ser. No. 09/657,421, filed on Sep. 8, 2000 and now issued as U.S. Pat. No. 6,626,180 each of which is hereby incorporated by reference into the present disclosure. In addition, the present application claims benefit and priority to U.S. Prov. Pat. Appl. No. 61/132,588, filed on Jun. 19, 2008 which is hereby incorporated by reference into the present disclosure.

and co-pending U.S. patent application Ser. No. 10/460,886, filed on Jun. 12, 2003 and now published as U.S. Pat. Pub. No. 2004/0250817, which is a continuation-in-part of U.S. patent application Ser. No. 09/657,421, filed on Sep. 8, 2000 and now issued as U.S. Pat. No. 6,626,180 each of which is hereby incorporated by reference into the present disclosure. In addition, the present application claims benefit and priority to U.S. Prov. Pat. Appl. No. 61/132,588, filed on Jun. 19, 2008 which is hereby incorporated by reference into the present disclosure.

### BACKGROUND OF THE INVENTION

1. Field of the Invention

The present invention relates to oral devices and methods and, more particularly, to apparatus and methods for inhibiting increases in lactate levels in individuals engaged in athletic activities, physical exertion, or other activities that may lead to anaerobic respiration.

2. Background of the Related Art

During times of physical and/or mental exertion the human body utilizes three primary pathways to create adenosine triphosphate (ATP), the necessary molecule for energy. Each method requires glucose and glycogen which are molecules obtained from carbohydrate ingestion. The quickest form of energy production is the creatine phosphate pathway but this produces very little ATP and only meets energy needs during the first few seconds of exercise. Another quick energy pathway is the glycolytic pathway which produces 3 ATP from 1 mole of glucose and 4 ATP from 1 mole of glycogen. This pathway lasts for 2-3 minutes so is not useful for long-term exercise. The final energy pathway is oxidative phosphorylation which produces 38 ATP from 1 mole of glucose and 39 ATP from 1 mole of glycogen. This is the preferred pathway when exercising aerobically as it sustains the body over several hours. All three pathways work together to create adequate ATP for energy needs during exercise.

The problem that arises for exercising individuals is that fatigue sets in and exercise must cease. There are a variety of reasons for this fatigue but one common reason, as supported by research, is the build up of lactic acid. Lactic acid itself does not seem to be the problem, but rather the by-products of lactic acid which are lactate and hydrogen ions. The accumulation of lactate and hydrogen occurs when persons begin to rely more heavily on the glycolytic pathway, with the end product of this pathway being lactic acid. For exercising individuals, the dependence on the glycolytic pathway occurs when exercise intensity increases, particularly at the end of a bout of exercise. Thus, the goal of many athletes is to decrease the amount of lactic acid production so that exercise intensity can be maintained throughout exercise, particularly competition. This would decrease fatigue and thereby increase performance. Therefore, a need exists for apparatus and methods that may assist in the reduction of lactate during athletic activities, physical training and other activities that may lead to increase lactate levels.

### SUMMARY OF THE INVENTION

Apparatus and methods in accordance with the present inventions may resolve one or more of the needs and shortcomings discussed above and may provide additional improvements and advantages as will be recognized by those skilled in the art upon review of the present disclosure.

The present inventions provide methods and apparatus for inhibiting the increase in lactate level in the human body's response to physical and/or mental exertion or other lactate generating activities without the use of pharmaceuticals. The methods and apparatus of the present inventions may be used to provide substantial reductions in lactate levels during and after physical and/or mental exertion. In one aspect, the physical and/or mental exertion may result from endurance training, such as running, walking, biking, rowing and swimming. Additional new indications for use of the apparatus and methods in accordance with the present inventions will be recognized by those skilled in the art upon review of the present disclosure. The present inventions provide methods for reducing lactate levels, inhibiting the increase of lactate levels or otherwise downregulating the normal increase in lactate levels in response to endurance training activities by using an oral apparatus. The apparatus may be worn during daily activities that can result in the production of lactate. The apparatus in accordance with the present inventions may also be worn during, before and/or after endurance training to manage the production of lactate.

In one aspect, a user may position an oral apparatus in accordance with the present inventions in his or her mouth before, during or after entering into a lactate generating activity such as, for example, endurance training. The oral apparatus include at least first bite pad and a second bite pad. The first bite pad and the second bite pad can be positioned on opposite sides of the user's mouth between at least two of the molars of a user.

In one aspect, the first bite pad and the second bite pad may generally configured to reduce or eliminate the accumulation of pressure at the temporomandibular joint as a user clenches his or her teeth and/or to reduce the pressure on the joint during normal daily activities of the user. When at least the molars of the user exert an opposing pressure on the first bite pad and the second bite pad, the lower mandible may be positioned to reduce the pressure at the temporomandibular joint relative to the pressures generated when a user is not wearing an oral apparatus. By reducing the pressure on the temporomandibular joint, a user may reduce the increase in lactate levels arising from their body's response to their activities. In one aspect, the pressure of at least the molars on the oral apparatus positions the lower mandible to generally reduce the pressure at the temporomandibular joint in an amount effective to downregulate the levels of lactate in the user. In another aspect, the pressure of at least the molars on the oral apparatus positions the lower mandible to reduce the pressure at the temporomandibular joint an amount sufficient to reduce the levels of lactate in the user. In yet another aspect, the pressure of at least the molars on the oral apparatus positions the lower mandible to inhibit an increase in lactate levels in the user. In another aspect, the pressure of at least the molars on the oral apparatus positions the mandibular condyles downward relative to the articular fossae at the temporomandibular joints a distance effective to regulate the levels of lactate in the user. In another aspect, the pressure of at least the molars on the oral apparatus positions the mandibular condyles downward relative to the articular fossae at the temporomandibular joints a distance effective to reduce the levels of lactate in the user. In yet another aspect, the pressure of at least the molars on the oral apparatus repositions the mandibular condyles downward relative to the articular fossae at the temporomandibular joints a distance effective to inhibit an increase in lactate levels in the user. In another aspect, the pressure the pressure of at least the molars on the oral apparatus positions the mandibular condyles downward and forward relative to the articular fossae at the temporomandibular joints at a distance effective to regulate the levels of lactate in the user. In another aspect, the pressure the pressure of at least the molars on the oral apparatus positions the mandibular condyles downward and forward relative to the articular fossae at the temporomandibular joints a distance effective to reduce the levels of lactate in the user. In yet another aspect, the pressure the pressure of at least the molars on the oral apparatus positions the mandibular condyles downward and forward relative to the articular fossae at the temporomandibular joints at a distance effective to inhibit an increase in lactate levels in the user. Thus, apparatus in accordance with the present invention may inhibit the increase in lactate the human body's response to physical and/or mental exertion or other lactate generating events.

Additionally or alternatively in another aspect, the oral apparatus may include at least first bite pad and a second bite pad generally configured to maintain or increase the patency of a user's airways. By maintaining the patency of a user's airway, a user may inhibit the increase in lactate levels arising from their body's response to their activities. Generally, the present apparatus and methods may prevent the positioning of the lower mandible in at least one orientation that decreases the patency of the user's airway by one or more of moving the jaw downward and slightly forward in response to efforts of the user to clench his or her teeth. In certain aspects, this positioning of the jaw may increase or maintain the patency of a user's airways as the user clenches his or her teeth. This maintenance or increase may improve the efficiency of a user's breathing thus reducing the production of lactate relative to a user not using an apparatus to reposition the jaw. Thus, apparatus in accordance with the present invention may inhibit the increase in lactate the human body's response to physical and/or mental exertion or other lactate generating events.

Other features and advantages of the invention will become apparent from the following detailed description, and from the claims.

### BRIEF DESCRIPTION OF THE FIGURES

FIGS. 1A and 1B illustrate the repositioning of a lower mandible with an exemplary oral apparatus to relieve pressure at the temporomandibular joint in accordance with aspects of the present invention;

FIG. 2A illustrates another exemplary apparatus in accordance with aspects of the present invention;

FIG. 2B illustrates the apparatus of FIG. 2A positioned in an exemplary manner to the lower mandible of a user;

FIG. 3A illustrates yet another exemplary apparatus in accordance with aspects of the present invention;

FIG. 3B illustrates the apparatus of FIG. 3A positioned in an exemplary manner on the maxilla of a user;

FIG. 4A illustrates yet another exemplary apparatus in accordance with aspects of the present invention;

FIG. 4B illustrates the apparatus of FIG. 4A positioned in an exemplary manner on the maxilla of a user;

FIG. 5A illustrates yet another exemplary apparatus in accordance with aspects of the present invention;

FIG. 5B illustrates the apparatus of FIG. 5A positioned in an exemplary manner to the lower mandible of a user.

FIG. 6A illustrates another exemplary apparatus in accordance with aspects of the present invention;

FIG. 6B illustrates the apparatus of FIG. 6A positioned in an exemplary manner on the maxilla of a user;

FIG. 7 illustrates another exemplary apparatus in accordance with aspects of the present invention;

FIG. 8A illustrates a perspective view of another exemplary apparatus in accordance with aspects of the present inventions;

FIG. 8B illustrates a bottom view of an exemplary apparatus similar to the appliance illustrated in FIG. 8A;

FIG. 8C illustrates a rear view of an exemplary apparatus similar to the appliance illustrated in FIG. 8A;

FIG. 9A illustrates a perspective view of an exemplary apparatus in accordance with aspects of the present inventions;

FIG. 9B illustrates a top view of an exemplary apparatus similar to the appliance illustrated in FIG. 9A; and

FIG. 9C illustrates a rear view of an exemplary apparatus similar to the appliance illustrated in FIG. 9A.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by one skilled in the art.

### DETAILED DESCRIPTION OF THE INVENTION

The present invention provides methods and oral apparatus for reducing the increase lactate levels typically associated with physical and/or mental exertion or other lactate generating activities. In certain aspects, the apparatus and methods may be particularly application to endurance training and activities, such as, for example walking, jogging, running, swimming, biking and the like. The figures generally illustrate embodiments of oral apparatus 60 for use in methods in accordance with the present invention, and results from testing the effect of the oral apparatus on lactate levels. The particular exemplary embodiments of the oral apparatus 60 illustrated in the figures have been chosen for ease of explanation and understanding of the implementation of various aspects of the present inventions. These illustrated embodiments are not meant to limit the scope of coverage but instead to assist in understanding the context of the language used in this specification and the appended claims. Accordingly, variations of oral apparatus 60 for use reducing lactate levels different from the illustrated embodiments may be encompassed by the appended claims.

Various exemplary embodiments of oral apparatus 60 in accordance with aspects of the present invention are illustrated throughout the figures for exemplary purposes. The illustrated oral apparatus 60 include at least a first bite pad 62 and a second bite pad 64. The oral apparatus 60 may adapted to be secured over one or both of the upper and lower teeth. The first bite pad 62 and the second bite pad 64 can be generally configured to one or more of position the lower mandible 42 relative to the maxilla 12 in a manner that relieves pressure at the temporomandibular joint 32 in response to the clenching of the user's teeth or move the lower mandible 42 downward and/or forward relative to the maxilla 12 as the user clenches his teeth preventing a reduction in the patency of the airway of a user usually associated with the clenching together of a user's teeth.

The first bite pad 62 and the second bite pad 64 are typically configured to be received between the occlusal surfaces of the upper (palatal) and lower (lingual) rows of teeth. Particularly, at least a portion of the first bite pad 62 and at least a portion of the second bite pad 64 are configured to be received between at least two molars. The first bite pad 62 and the second bite pad 64 are generally sized to be received between the occlusal surfaces of at least two teeth (one upper and one lower). In one aspect, the first bite pad 62 and the second bite pad 64 may have dimensions of about 5/16 inches wide and about 1⅛ inches long. When positioned between the occlusal surfaces of the upper and lower teeth, the first bite pad 62 and the second bite pad 64 are typically configured to space the upper and lower molars apart by 0.5 millimeters and 4.5 millimeters at the posterior edge 72 of each of the bite pads 62, 64. In certain aspects, the first bite pad 62 and the second bite pad 64 can be configured to space the upper and lower molars apart by 1.00 millimeters and 4.00 millimeters at the posterior edge 72 of each of the bite pads 62, 64. In one particular embodiment, the first bite pad 62 and the second bite pad 64 can be configured to space the upper and lower molars apart by 2.0 millimeters at the posterior edge of each of the bite pads. The first bite pad 62 and the second bite pad 64 may have a uniform thickness from anterior edge to posterior edge and medial edge to lateral edge. The first bite pad 62 and the second bite pad 64 may also have a variable thickness between one or more of the anterior edge 70 to posterior edge 72 and medial edge to lateral edge. In one aspect, the first bite pad 62 and the second bite pad 64 can have one or more of a planar upper surface and a planar lower surface. In one aspect of a variable thickness, the first bite pad 62 and the second bite pad 64 may be configured to space the upper and lower teeth apart by 0.0 millimeters and 4.5 millimeters at the anterior edge of each of the bite pads. In certain aspects, the first bite pad 62 and the second bite pad 64 are configured to space the upper and lower teeth apart by 0.2 millimeters and 2.00 millimeters at the anterior edge of each of the bite pads. In one particular embodiment, the first bite pad 62 and the second bite pad 64 are typically configured to space the upper and lower teeth apart by 1.0 millimeters at the anterior edge of each of the bite pads. When the anterior edge and the posterior edge of the first bite pad 62 and the second bite pad 64 are of different thicknesses, the first bite pad 62 and the second bite pad 64 may have the shape, in profile, of a wedge. The wedge is typically thinner in anterior edge 70 and thicker at the posterior edge 72.

Without being bound by theory, the oral apparatus 60 may alter the central nervous system's afferent input by relieving pressure at the temporomandibular joint, either through periodontal ligament or temporomandibular joint pathways, ultimately affecting the hypothalamic-pituitary-adrenal (HPA) axis, ultimately altering lactate production. Alternatively or additionally, the oral apparatus 60 may maintain the patency of the user's airway as the patency of a user's airway is normally reduced during clenching of teeth or otherwise when the teeth are similarly juxtaposed. Thus, an oral apparatus 60 in accordance with the present inventions may position the lower mandible 42 downward and/or forward when the user attempts to clench his or her teeth preventing a reduced patency in the adjacent airway and ultimately increasing the efficiency of breathing which normally reduces the rate of lactate production.

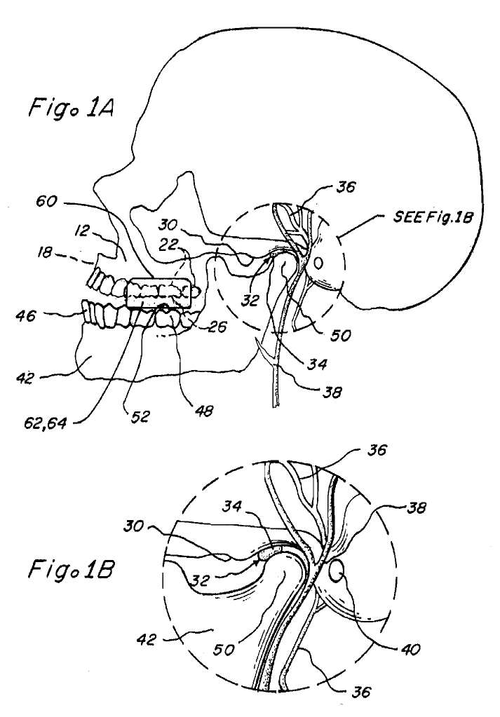

FIGS. 1A and 1B, show an oral apparatus 60 positioned within the skull of a user. The lower mandible 42 articulates with the skull at the temporomandibular joint 32. The maxilla 12 supports upper anterior teeth (incisors and canines) 18 and upper posterior teeth (molars and bicuspids) 22 which have cusps and occlusal surfaces 26. The lower mandible 42 supports the lower anterior teeth (incisors and canines) 46 and lower posterior teeth (molars and bicuspids) 48 with occlusal surfaces 52. The mandibular condyles 50 of the lower mandible 42 are received within the articular fossae 30 with each side supported and cushion by a meniscus 34 to form the temporomandibular joint 32. When the mouth opens, two distinct motions typically occur at the temporomandibular joint 32. The first motion is rotation around a horizontal axis through the heads of mandibular condyle 50. The second motion is translation. When the mouth is fully open, the mandibular condyle 50 may lie beneath the anterior band of the meniscus 34. The mandibular condyle 50 and meniscus 34 both move together anteriorly beneath the articular eminence 30. In the closed position, the thick posterior band of the meniscus 34 lies immediately above the mandibular condyle 50. As the mandibular condyle 50 translates forward, the thinner intermediate zone of the meniscus 34 becomes the articulating surface between the mandibular condyle 50 and the articular eminence 30.

The first bite pad 62 and the second bite pad 64 of oral apparatus 60 in accordance with aspects of the present inventions are typically positioned between at least the upper molars and/or bicuspids 22 and the lower molars and/or bicuspids 48 to prevent excessive pressure, reduce pressure or to eliminate pressure at the temporomandibular joint 32 as the lower mandible 42 is drawn toward the maxilla 12 during clenching and/or to prevent a reduction or increase the patency of the adjacent airway during clenching. As will be recognized by those skilled in the art upon review of the presents disclosure, the first bite pad 62 and the second bite pad 64 may be generally configured to direct the posterior portion of the mandible 42 downward and/or forward when a user applies a clenching force on the bite pads 62, 64 with his or her molars.

In certain aspects, the oral apparatus 60 may withdraw the mandibular condyle 50 downward from the articular fossa 30 in response to an attempt by a user to clench their teeth. In other aspects, the oral apparatus 60 may withdraw the mandibular condyle 50 downward and forward from the articular fossa 30 in response to an attempt by a user to clench their teeth. Such movement may relieve or create pressure on a number of vessels in the area of the temporomandibular joint 32. Particularly, the auriculo-temporalis nerve 36 and the supra-temporal artery pass posterior to the temporomandibular joint 32. The auriculo-temporalis nerve 36 functions in both sensory and motor activities of the body. The supra-temporal artery 38 provides blood circulation to the head. Impingement, pinching, rupture or blockage of the supra-temporal artery 38 may restrict blood flow to the brain. Additional nerve and vessels are also in close proximity to the temporomandibular joint and may be affected by a repositioning of the lower mandible 42.

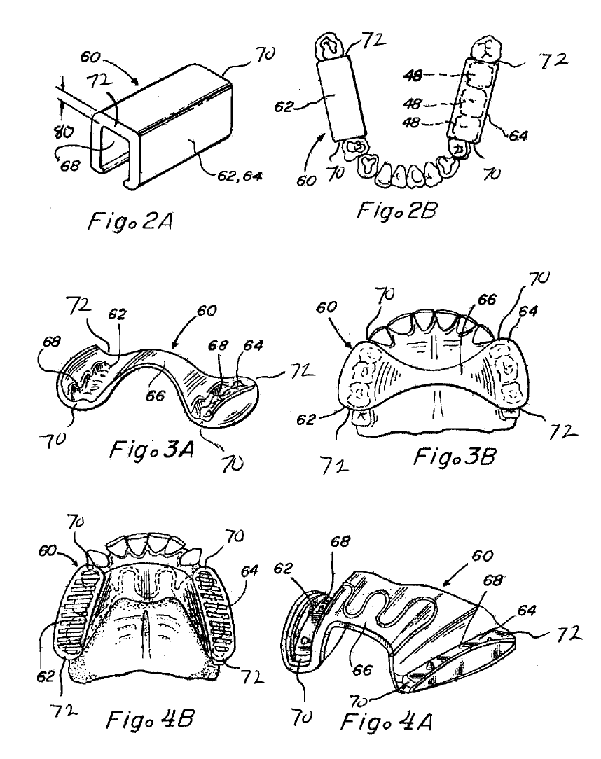

FIGS. 2A and 2B illustrate an exemplary embodiment for an oral apparatus 60 in accordance with aspects of the present inventions. The illustrated oral apparatus 60 includes a first bite pad 62 and a second bite pad 64. Each of the first bite pad 62 and the second bite pad 64 of the illustrated embodiment are configured to be received between at least one of the upper teeth and at least one of the lower teeth of a user. The first bite pad 62 and the second bite pad 64 may be configured to be received over at least one of the upper or lower teeth of a user. The first bite pad 62 and the second bite pad 64 each define a channel 68 to receive the teeth of the user. As particularly illustrated, the first bite pad 62 and the second bite pad 64 are configured to be received over the lower molar 48. Further, the first bite pad 62 and the second bite pad 64 may be configured to be secured to one of the upper or the lower teeth of a user to retain the first bite pad 62 and the second bite pad 64 at the desired location. A thickness 80, size and shape of the portion of each bite pad 62, 64 received between the teeth of the user determines how the lower mandible 42 will be positioned when a user clenches his/her teeth as will be recognized by those skilled in the art upon review of the present disclosure.

FIGS. 3A and 3B illustrate another exemplary embodiment for an oral apparatus 60 in accordance with aspects of the present inventions. Similar oral apparatus 60 are disclosed in U.S. Pat. No. 6,553,996 the disclosure of which is hereby incorporated by reference in its entirety. The illustrated oral apparatus 60 includes a first bite pad 62 and a second bite pad 64 secured to one another by an interconnect 66 configured to be positioned adjacent to the roof of a user's mouth. The exemplary interconnect 66 is configured to extend across a substantially central portion of the roof of the user's mouth. Each of the first bite pad 62 and the second bite pad 64 of the illustrated embodiment are configured to be received between at least one of the upper teeth and at least one of the lower teeth of a user. The first bite pad 62 and the second bite pad 64 of the illustrated embodiment may be configured to be received over at least one of the upper or lower teeth of a user. The first bite pad 62 and the second bite pad 64 each define a channel 68 to receive the teeth of the user. As particularly illustrated, the first bite pad 62 and the second bite pad 64 are configured receive the anterior two upper molars 22 on each side of a users mouth and the bicuspids 22 of the user. The channels 68 of each of the first bite pad 62 and the second bite pad 64 have been shaped to conform to the molars 22, 48 of a user. A thickness, size and shape of the portion of each bite pad 62, 64 received between the teeth of the user determines how the lower mandible 42 will be positioned when a user clenches his/her teeth as will be recognized by those skilled in the art upon review of the present disclosure.

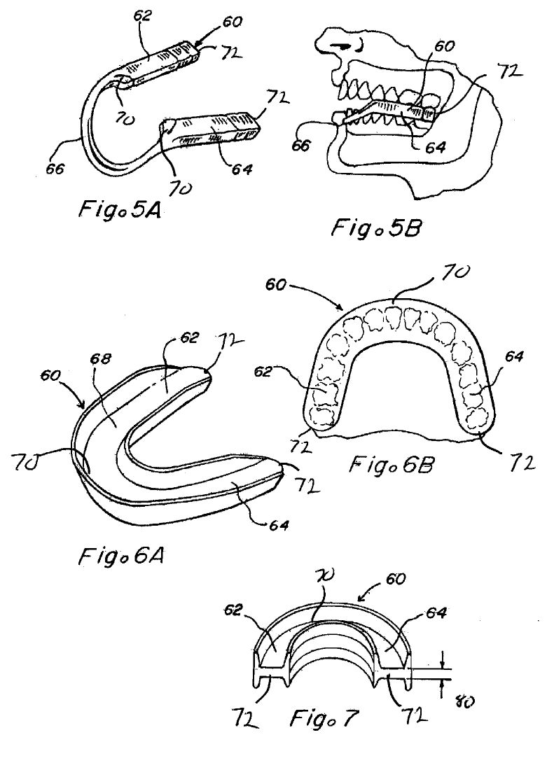

FIGS. 4A and 4B illustrate another exemplary embodiment for an oral apparatus 60 in accordance with aspects of the present inventions. Similar oral apparatuses 60 are disclosed in U.S. Pat. No. 6,415,794 the disclosure of which is hereby incorporated by reference in its entirety. The illustrated oral apparatus 60 includes a first bite pad 62 and a second bite pad 64 secured to one another by an interconnect 66 configured to be positioned adjacent to an anterior portion of the roof of a user's mouth. Each of the first bite pad 62 and the second bite pad 64 of the illustrated embodiment are configured to be received between at least one of the upper teeth and at least one of the lower teeth of a user. The first bite pad 62 and the second bite pad 64 of the illustrated embodiment may be configured to be received over at least one of the upper or lower teeth of a user. The first bite pad 62 and the second bite pad 64 each define a channel 68 to receive the teeth of the user. As particularly illustrated, the first bite pad 62 and the second bite pad 64 are configured receive the all of the upper molars 22 and the upper bicuspids 22 of a user. The channels 68 of each of the first bite pad 62 and the second bite pad 64 may be sized to securely receive the molars 22, 48 of a user. A thickness 80, size and shape of the portion of each bite pad 62, 64 received between the teeth of the user determines how the lower mandible 42 will be positioned when a user clenches his/her teeth as will be recognized by those skilled in the art upon review of the present disclosure.

FIGS. 5A and 5B illustrate another exemplary embodiment for an oral apparatus 60 in accordance with aspects of the present inventions. Similar oral apparatuses 60 are disclosed in U.S. Pat. No. 6,371,758 the disclosure of which is hereby incorporated by reference in its entirety. The illustrated oral apparatus 60 includes a first bite pad 62 and a second bite pad 64 secured to one another by an interconnect 66 configured to be positioned between the lower lip and the lower teeth and/or gums of a user. The first bite pad 62 and the second bite pad 64 may be configured to be received between the upper or lower teeth 22, 48 of a user. The first bite pad 62 and the second bite pad 64 each define an upper and a lower surface. The upper surface configured to contact the upper teeth 22 of the user. The lower surface configured to contact the lower teeth 48 of the user. As particularly illustrated, the first bite pad 62 and the second bite pad 64 are configured contact the all of the upper molars 22 and upper bicuspids 22 and the lower molars 48 and lower bicuspids 48. A thickness 80, size and shape of each bite pad 62, 64 and the associated shape of the upper and lower surfaces where received between the teeth of the user determines how the lower mandible 42 will be positioned when a user clenches his/her teeth as will be recognized by those skilled in the art upon review of the present disclosure.

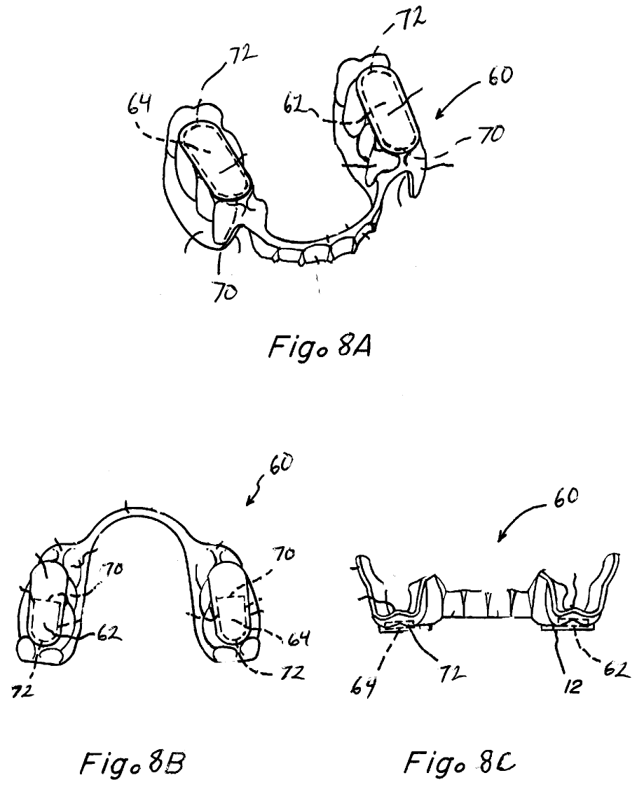

FIGS. 6A and 6B illustrate another exemplary embodiment for an oral apparatus 60 in accordance with aspects of the present inventions. Similar oral apparatuses 60 are disclosed in U.S. Pat. No. 6,691,710 the disclosure of which is hereby incorporated by reference in its entirety. The illustrated oral apparatus 60 includes a first bite pad 62 and a second bite pad 64 secured to one another by an interconnect 66 configured to be positioned over the incisors and canines of a user. Each of the first bite pad 62 and the second bite pad 64 may be configured to be received between the upper or lower teeth of a user. The first bite pad 62 and the second bite pad 64 define a channel 68 which is continuous through the interconnect 66 to receive the teeth of the user. As particularly illustrated, channel 68 extends through the first bite pad 62, the interconnect 66, and the second bite pad 64 receive all of the upper teeth of a user. The channel 68 has been sized to receive the upper teeth of the user and may be configured to securely hold the teeth of the user. A thickness 80, size and shape of the portion of each bite pad 62, 64 received between the teeth of the user determines how the lower mandible 42 will be positioned when a user clenches his/her teeth as will be recognized by those skilled in the art upon review of the present disclosure.

FIG. 7 illustrates another exemplary embodiment for an oral apparatus 60 in accordance with aspects of the present inventions. The illustrated oral apparatus 60 includes a first bite pad 62 and a second bite pad 64 secured to one another by an interconnect 66 configured to be positioned over the incisors and canines of a user. Each of the first bite pad 62 and the second bite pad 64 of the illustrated embodiment are configured to be received between at least one of the upper teeth and at least one of the lower teeth of a user. The first bite pad 62 and the second bite pad 64 define a channel 68 and a lower channel 69. Each of the channel 68 and the lower channel 69 is shown as continuously extending through the interconnect 66. The channel 68 is configured to receive the upper teeth of the user. The lower channel 69 is configured to receive the lower teeth of the user. As particularly illustrated, each of channel 68 and lower channel 69 extends through all of the first bite pad 62, the interconnect 66, and the second bite pad 64 receive all of the teeth of a user. Each of the channel 68 and lower channel 69 has been sized to receive the teeth of the user and at least one of the channel 68 and the lower channel 69 may be configured to securely hold the teeth of the user. A thickness 80, size and shape of the portion of each bite pad 62, 64 received between the teeth of the user determines how the lower mandible 42 will be positioned when a user clenches his/her teeth as will be recognized by those skilled in the art upon review of the present disclosure.

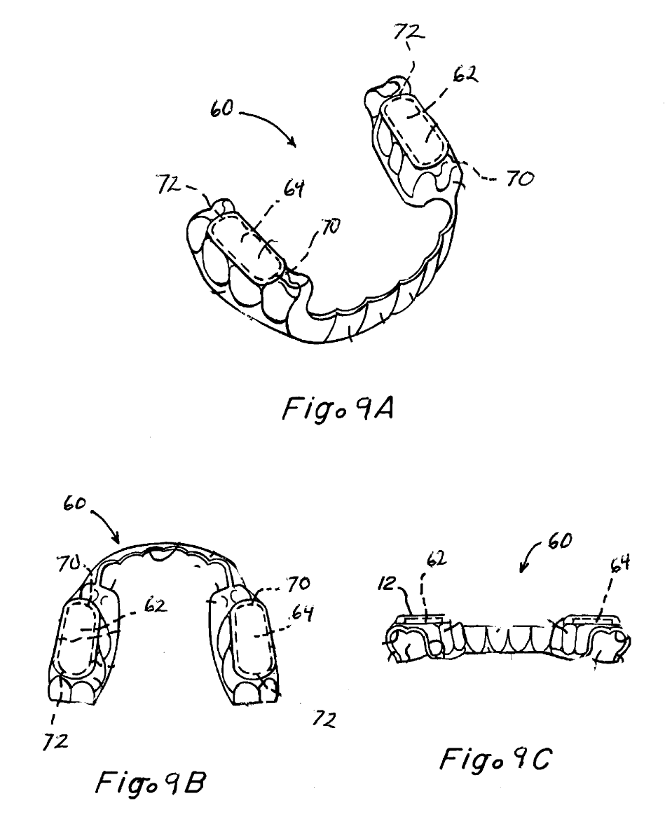

FIGS. 8A and 9A illustrate perspective views of exemplary embodiments of an oral apparatus 60 configured to be positioned over the teeth of a user in accordance with aspects of the present inventions. As illustrated, the embodiments of oral apparatus 60 may be configured to be received over at least some of the upper teeth or lower teeth of a user.

As illustrated, the body of the oral apparatus 60 defines a first bite pad 62, a second bite pad 64 secured to one another by a connector band. The connector band may be configured to be positioned against any of the lingual, the palatal, the labial or the buccal surface of the gums and/or teeth. The first bite pad 62 and the second bite pad 64 are generally configured to maintain a desired thickness during clenching to facilitate the movement of lower mandible downward and/or forward. The first bite pad 62 and the second bite pad 64 are generally configured to be positioned over at least one of the lower molars on each side of a user's jaw when worn by a user. When the upper teeth and the lower teeth of a user are juxtaposed, the first bite pad 62 and the second bite pad 64 may be positioned between at least some of the upper molars and/or premolars and some of the lower molars and/or premolars.

The connector band connects the first bite pad 62 to the second bite pad 64. In certain embodiments, the connector band is typically integrally formed with or secured to a body that may function to secure the first bite pad 62 and the second bite pad 64 to the teeth of a user. The connector band may be configured to extend as an arch around the lingual and/or palatal sides of at least the front teeth of a user.

The first bite pad 62 and second bite pad 64 may have a unitary structure or a component of a composite structure. When the bite pads 62, 64 may be embedded or encapsulated within one or more materials forming the oral apparatus 60 as illustrated in FIGS. 8A to 9C. The first bite pad 62 and second bite pad 64 are typically formed from a material having a greater hardness than the material of any over layered material forming the oral apparatus 60. The first bite pad 62 and second bite pad 64 are typically of sufficient hardness to resist substantial penetration by the teeth and substantial deformation as the teeth of a user are clenched. A material such as high density polyethylene or polypropylene may be used as well as other materials that will be recognized by those skilled in the art upon review of the present disclosure. In various aspects, the first bite pad 62 and second bite pad 64 may be configured to at least one of guide the lower mandible 42 into a desired position that may reduces or alleviates pressure at the temporomandibular joint or maintain or increase patency of the user's airway during clenching. Accordingly, the first bite pad 62 and second bite pad 64 typically will have a durometer of between about 60 D to about 90 D although this may vary in certain embodiments of the invention to overcome the mechanics of the user's anatomy. The first bite pad 62 and second bite pad 64 may have a constant thickness from the anterior edge 70 to the posterior edge 72, they may be thicker at the anterior edge 70 and be tapered to a thinner posterior edge 72, they may have a thinner anterior edge 70 and be tapered to a thicker posterior edge 72, or they may be otherwise configured as will be recognized by those skilled in the art upon review of the present disclosure to position the lower mandible 12 in accordance with aspects of the present inventions. The oral apparatus 60 are generally configured to position the first bite pad 62 and the second bite pad 64 over the occlusal surfaces of at least one the premolars and at least one of the molars. In other aspects, the first bite pad 62 and the second bite pad 64 may be solely positioned over the occlusal surfaces of one or more molars.

FIGS. 8B and 8C illustrate various features of an embodiment of an oral apparatus 60 of FIG. 8A. As illustrated, one or more molars and premolars may be positioned within the channel formed to position the first bite pad 62 and second bite pad 64 over the teeth of a user. The connector band is configured to extend around the front of the mouth along the palatal surface of incisors and canines. The connector band forms an arch between the first bite pad 62 and second bite pad 64 which is received against a palatal surface of at least the incisors and canines of a user.

FIGS. 9B and 9C illustrate various features of an embodiment of an oral appliance 10 for positioning on lower teeth of a user. As illustrated, one or more molars and premolars may be positioned within the channels formed below each of the first bite pad 62 and second bite pad 64. The connector band is configured to extend around the front of the mouth along the palatal surface of incisors and canines. The connector band forms an arch between the first bite pad 62 and second bite pad 64 which is received against a palatal surface of at least the incisors and canines of a user.

In use, a first bite pad 62 and a second bite pad 64 of an oral apparatus 60 is positioned between the occlusal surfaces of the upper and lower teeth on both the left and right side of the user's mouth. The bite pads 62, 64 are positioned between at least two molars (one upper and one lower) on each side of the user's mouth. The bite pads 62, 64, in various other embodiments, may be positioned between at least the second premolar and first molar, may extend between at least the second premolar and second molar, may extend between at least the first premolar and second molar and may extend between at least the canines and the third molar. A clenching force is exerted between at least the molars of the user against bite pads 62, 64. The lower mandible 42 is directed at least one of downward and forward from the contact of the molars with bite pads 62, 64 of the oral apparatus 60. In one aspect, one or more of the downward and forward movement of the lower mandible 42 reduces and/or eliminates pressure between the mandibular condyle 50 articular fossa at the temporomandibular joint as the teeth of the user are clenched. In another aspect, one or more of the downward and forward movement of the lower mandible 42 reduces and/or eliminates a reduction in the patency of the portion of the airway adjacent the lower mandible 42 when the teeth of the user are clenched.

The apparatus and methods of the present invention are shown to position the lower mandible in a position effective to inhibit an increase in lactate levels or otherwise down-regulate lactate levels in response to endurance training. In accordance with aspects of the present inventions, the oral apparatus 60 may be used be in endurance training activities, such as running, walking, biking, rowing and swimming (including stationary biking and running) to reduce or inhibit lactate production. The oral apparatus 60 may in certain aspects be positioned in the mouth prior to endurance training to reduce an associated increase in lactate levels. In other aspects, the oral apparatus 60 may be positioned in the mouth of a user after or during endurance training to reduce an increase in lactate levels. The oral apparatus 60 may be worn before, during and after physical and/or mental activities sufficient to produce lactate to reduce the accumulation of lactate from the activities.

### Example 1

In one study, 24 subjects, between the ages of 18 and 24 years old, were recruited to participate in this study via flyers and “word of mouth.” Subjects were asked to complete a treadmill protocol in which they exercised at 75-85% of their maximal heart rate for 30 minutes at 0% elevation. Participants were asked to complete this exercise protocol on two separate days. In one trial, subjects completed the protocol without the use of a mouthpiece and in the other trial the subjects completed the protocol with the use of a mouthpiece. Timing of mouthpiece use was randomly selected before testing began. Pre, during and post exercise, subjects were asked to give a finger prick sample of blood to evaluate lactate levels. An Accutrend Lactate analyzer (Roche Diagnostics GmbH, Mannheim, Germany) was used to assay the drop of blood and results were recorded at each testing. In addition to lactate assays, subjects' heart rates were evaluated before, during, and after each exercise trial using a Polar Heart Rate Monitor (Warminster, Pa.).

Results indicated that there was a significant improvement (p value<0.05) in lactate levels at the 30 minute exercise mark of the testing (p value=0.024). The mean lactate levels at 30 minutes with the mouth piece were 4.01 mmol/L and 4.83 mmol/L without the use of the mouth piece.

### Example 2

In another study, 18 subjects, between the ages of 18 and 24 years old, were recruited to participate in this study via “word of mouth.” Subjects were asked to complete a treadmill protocol in which they exercised at 75-85% of their maximal heart rate for 30 minutes at 0% elevation. Participants were asked to complete this exercise protocol on two separate days. In one trial, subjects completed the protocol without the use of a mouthpiece and in the other trial the subjects completed the protocol with the use of a mouthpiece. Timing of mouthpiece use was randomly selected before testing began. Pre, 15 minutes and immediately following exercise, subjects were asked to give a finger prick sample of blood to evaluate lactate levels. An Accutrend Lactate analyzer (Roche Diagnostics GmbH, Mannheim, Germany) was used to assay the drop of blood and results were recorded at each testing.

Results indicated that there was a significant improvement (p value<0.05) in lactate levels at the 30 minute exercise mark of the testing (p value=0.01). The mean lactate levels at 30 minutes with the mouth piece were 3.79 mmol/L and 4.85 mmol/L without the use of the mouth piece.

The foregoing discussion discloses and describes merely exemplary embodiments of the present invention. Upon review of the specification, one skilled in the art will readily recognize from such discussion, and from the accompanying drawings and claims, that various applications, changes, modifications and variations can be made therein without departing from the spirit and scope of the invention as defined in the following claims. A number of patents and applications have been incorporated by reference in the present disclosure, any inconsistencies between the substance or terminology of these referenced disclosures and the present application should be interpreted in a manner consistent solely with the present disclosure.

What is claimed is:

1. A method for inhibiting lactate accumulation, comprising:
positioning an oral apparatus having at least first bite pad and a second bite pad between at least the molars of a user, where each of the first bite pad and the second bite pad comprise a posterior edge having a thickness greater than the anterior edge;
applying a clenching force between the molars against a least a portion of the first bite pad and the second bite pad; and
positioning a lower mandible of the user downward and forward relative to a maxilla of the user with the clenching force applied by the user.

2. The method of claim 1, further comprising reducing the pressure at the temporomandibular joint.

3. The method of claim 1, further comprising positioning a mandibular condyle downward relative to an articular fossa at the temporomandibular joint a distance effective to down regulate the production of lactate in the user.

4. The method of claim 1, further comprising positioning a mandibular condyle downward relative to an articular fossa at the temporomandibular joint a distance effective to reduce the levels of lactate in the user.

5. The method of claim 1, further comprising positioning a mandibular condyle downward relative to an articular fossa at the temporomandibular joint a distance effective to inhibit an increase in lactate levels in the user.

6. The method of claim 1, further comprising positioning a mandibular condyle downward and forward relative to an articular fossa at the temporomandibular joint a distance effective to regulate the levels of lactate in the user.

7. The method of claim 1, further comprising positioning a mandibular condyle downward and forward relative to an articular fossa at the temporomandibular joint a distance effective to inhibit an increase in lactate levels in the user.

8. The method of claim 1, further comprising positioning the lower mandible downward and forward a distance effective to down regulate the production of lactate in the user.

9. The method of claim 1, further comprising positioning the lower mandible downward and forward a distance effective to inhibit an increase in lactate levels in the user.

10. The method of claim 1, further comprising positioning the lower mandible downward and forward a distance effective to reduce the levels of lactate in the user.

11. The method of claim 1, further comprising positioning the lower mandible downward and forward a distance effective to regulate the levels of lactate in the user.
